(19) United States
(12) Patent Application Publication
(10) Pub. No.: US 2024/0415600 A1
(43) Pub. Date: Dec. 19, 2024

(54) MONITORING UTILIZATION OF REUSABLE SURGICAL DEVICES

(71) Applicant: Alcon Inc., Fribourg (CH)

(72) Inventor: Kevin Canzone, Coto de Caza, CA (US)

(21) Appl. No.: 18/731,556

(22) Filed: Jun. 3, 2024

Related U.S. Application Data
(60) Provisional application No. 63/508,287, filed on Jun. 15, 2023.

(51) Int. Cl. A61B 90/00; A61B 90/98; G16H 40/40

(52) U.S. Cl. CPC A61B 90/08; A61B 90/98; G16H 40/40; A61B 2090/0803; A61B 2090/0807; A61B 2090/0814

(57) ABSTRACT

Certain aspects of the present disclosure provide systems and methods for monitoring utilization of reusable surgical devices in a surgical operating environment. In certain embodiments, a method may be performed by a surgical console. The method includes receiving an indicator of an intent to use a reusable surgical device in a surgical procedure. The method also includes, responsive to the received indicator, receiving, from memory associated with the reusable surgical device, a usage value associated with an amount of previous usage of the reusable surgical device. The method also includes assessing an acceptability of the reusable surgical device for use in the surgical procedure based on a comparison of the usage value with a usage threshold associated with a usage limit of the reusable surgical device. The method also includes allowing or denying usage of the reusable surgical device in the surgical procedure based on the assessed acceptability.

### BACKGROUND

Many surgical devices can be safely reused in multiple surgical procedures. The ability to reuse surgical devices is usually limited, however. Since surgical devices frequently move between different surgical environments and systems, utilization can be difficult to track.

### SUMMARY

The present disclosure relates to surgical systems and methods, and more particularly, to systems and methods for monitoring utilization of reusable surgical devices.

In certain embodiments, one general aspect includes a method of monitoring utilization of reusable surgical devices in a surgical operating environment. The method may be performed by a surgical console. The method includes receiving an indicator of an intent to use a reusable surgical device in a surgical procedure. The method also includes, responsive to the received indicator, receiving, from memory associated with the reusable surgical device, a usage value associated with an amount of previous usage of the reusable surgical device. The method also includes assessing an acceptability of the reusable surgical device for use in the surgical procedure based on a comparison of the usage value with a usage threshold associated with a usage limit of the reusable surgical device. The method also includes allowing or denying usage of the reusable surgical device in the surgical procedure based on the assessed acceptability of the reusable surgical device.

In certain embodiments, another general aspect includes a system for monitoring utilization of reusable surgical devices in a surgical operating environment. The system includes a reader and a surgical console and communicably coupled to the reader. The surgical console is operable to receive an indicator of an intent to use a reusable surgical device in a surgical procedure. The surgical console is also operable, responsive to the received indicator, to receive, via the reader, from memory associated with the reusable surgical device, a usage value associated with an amount of previous usage of the reusable surgical device. The surgical console is further operable to assess an acceptability of the reusable surgical device for use in the surgical procedure based on a comparison of the usage value with a usage threshold associated with a usage limit of the reusable surgical device. In addition, the surgical console is operable to allow or deny usage of the reusable surgical device in the surgical procedure based on the assessed acceptability of the reusable surgical device.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an operating environment with various surgical devices and systems, in accordance with certain embodiments of the present disclosure.

FIG. 2 illustrates certain aspects of the operating environment of FIG. 1, in accordance with certain embodiments of the present disclosure.

FIG. 3 illustrates how various components of the operating environment, shown in FIGS. 1-2, communicate and operate together, in accordance with certain embodiments of the present disclosure.

FIG. 4 illustrates an example of a process for monitoring utilization of surgical devices, in accordance with certain embodiments of the present disclosure.

FIG. 5 illustrates an example of a process for monitoring utilization of surgical devices via usage values, in accordance with certain embodiments of the present disclosure

To facilitate understanding, identical reference numerals have been used, where possible, to designate identical elements that are common to the Figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

The number and complexity of surgical procedures, including ophthalmic procedures such as vitreoretinal surgery, continues to increase every day. Various surgical devices, including surgical tools such as ophthalmic handpieces and their components, may be utilized to perform the procedures. Although some surgical devices can be sterilized and safely reused, there comes a time when even reusable devices are no longer suitable for surgical use and should be discarded and/or replaced. In certain embodiments, it would be advantageous, therefore, to monitor parameters related to device utilization, such as shelf life, usage amount, and/or the like. Problematically, however, surgical devices often move between different surgical environments and systems, thus making tracking difficult. The problem is compounded by the fact that surgical devices are used in sterile environments. As a result, contact-based tracking methods are generally unsuitable due to contamination risk.

One approach to the above problem might be to use a packaging label or mark, such as a barcode or quick response (QR) code, to provide certain device information for tracking purposes. However, since packaging labels and marks are not generally updatable, such methods would be limited to providing static information, such as an identifier. Such a tracking or monitoring system would be reliant upon device-specific data being stored and updated elsewhere in relation to whatever identifier is provided, which poses a challenge as the device travels between environments and systems.

The present disclosure describes examples of monitoring the utilization of reusable surgical devices, for example, by maintaining device-specific profile data in memory embedded in, coupled to, or otherwise natively associated with the devices. The memory can reside in radio frequency identification (RFID) or near-field communication (NFC) tags, for example, so as to enable non-contact access. In various embodiments, a computer associated with a surgical operating environment, such as a surgical console or a component thereof, can assess an acceptability of a given surgical device at or near the time of a surgical procedure, for example, based on device-specific profile data retrieved from memory associated with the device. In various embodiments, the computer can deny the device's usage in the surgical procedure if it determines that the device is unacceptable, for example, due to diminished safety, utility, and/or the like. In various embodiments, the computer can update the device-specific data to include new information, such as data reflective of the device's usage in the surgical procedure at issue. Accordingly, surgical use of the device can be tracked and the device can be discarded and/or replaced when its usage limit is reached.

Advantageously, in certain embodiments, systems described herein can monitor utilization of reusable surgical devices without any external storage of device-specific profile data, such as in network, system, or cloud storage locations. In certain embodiments, since the device-specific profile data is stored and maintained in memory natively associated with the devices, such data travels with the devices as they move between different environments and systems. Further, in embodiments in which RFID, NFC, or similar protocols are utilized for data transfer, the device-specific data can be retrieved and updated without contamination risk to the devices, thus rendering the method suitable for sterile environments such as those involving sterile surgical ophthalmic devices.

As used herein, the term “surgical system” may refer to any surgical system, console, or device for performing a surgical procedure. For example, the term “surgical system” may refer to a surgical console, such as a phacoemulsification console, a vitrectomy console, a laser system, or any other consoles, systems, or devices used in an ophthalmic operating room, as known to one of ordinary skill in the art. Note that although certain embodiments herein are described in relation to ophthalmic systems, devices, and environments, the embodiments described herein are similarly applicable to other types of medical or surgical systems, devices, and environments.

As used herein, the term “sensor” may refer to any type of device that detects or measures, e.g., a physical input, and records, indicates, or otherwise responds to the physical input. For example, the term “sensor” may refer to a device configured to detect or measure a position, location, proximity (e.g., to a surgical console), tilt, height, speed (e.g., an accelerometer), temperature, etc., of a surgical device, system, or user. In certain examples, the term “sensor” may refer to a device configured to detect touch, i.e., touching of a surgical device or system by a user, such as a capacitive- or resistive-type touch sensor. In certain examples, the term “sensor” may refer to an imaging device configured to detect and relay image-based information, such as a charge-coupled device (CCD) or an active-pixel sensor (APS), such as a complementary metal-oxide-semiconductor (CMOS) sensor.

Although generally described with reference to ophthalmic surgical devices and systems, the devices and systems described herein may be implemented with other devices and systems, such as devices and systems for other surgeries, without departing from the scope of the present application.

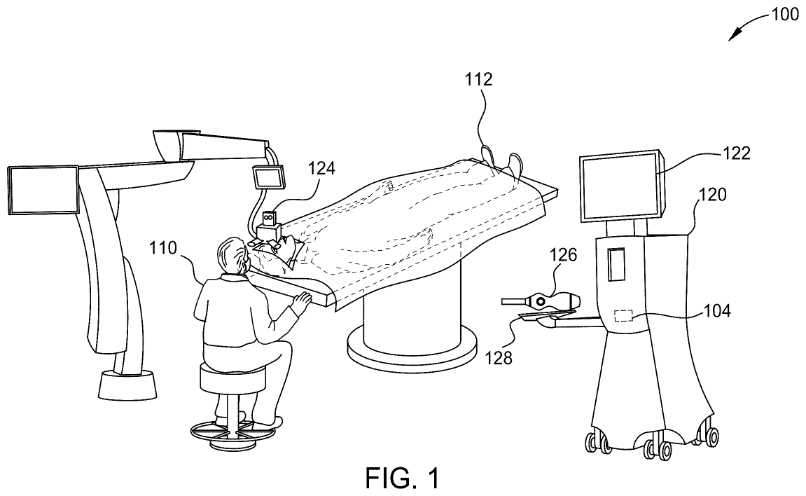

FIG. 1 illustrates an example of an operating environment 100, such as an ophthalmic operating environment, in which a surgical console 120 may be utilized for performance of a surgical procedure, according to embodiments of the present disclosure. As shown, the operating environment 100 further includes a surgeon 110, a patient 112, as well as a plurality of surgical systems and devices, such as a surgical console 120 having a display device 122, a microscope system 124, a surgical tool 126, and a surgical tray 128. Generally, examples of suitable surgical systems that may be included in the operating environment 100 include surgical devices and consoles for performing vitreoretinal procedures, cataract surgeries, corneal transplants, glaucoma surgeries, LASIK (Laser-Assisted In Situ Keratomileusis) surgeries, refractive lens exchanges, trabeculectomies, keratotomy procedures, and keratoplasty surgeries, or other devices and consoles identifiable by those of ordinary skill. Consoles that are capable of performing two or more of these procedures are also within the scope of this disclosure. An example of a console configured for performing vitreoretinal procedures is the Constellation® System available from Alcon Laboratories, Inc., Fort Worth, Texas. An example of a console configured for performing cataract surgeries is the Centurion® System available from Alcon Laboratories, Inc., Fort Worth, Texas.

The surgical console 120 includes a controller 104. The controller 104 is configured to cause the surgical console 120 to perform one or more tasks for driving a surgical tool, e.g., the surgical tool 126. In the example of FIG. 1, the controller 104 is integrated within the surgical console 120, where the controller 104 includes or refers to one or more processors and/or memory devices integrated within the surgical console 120. In certain other embodiments, the controller 104 is a stand-alone device or module that is in wireless or wired communication with, e.g., the surgical console 120 and other devices within the operating environment 100. In certain embodiments, the controller 104 refers to a set of software instructions that a processor associated with the surgical console 120 is configured to execute. In certain aspects, operations of the controller 104 may be executed partly by the processor associated with controller 104 and/or the surgical console 120 and partly in a public or private cloud

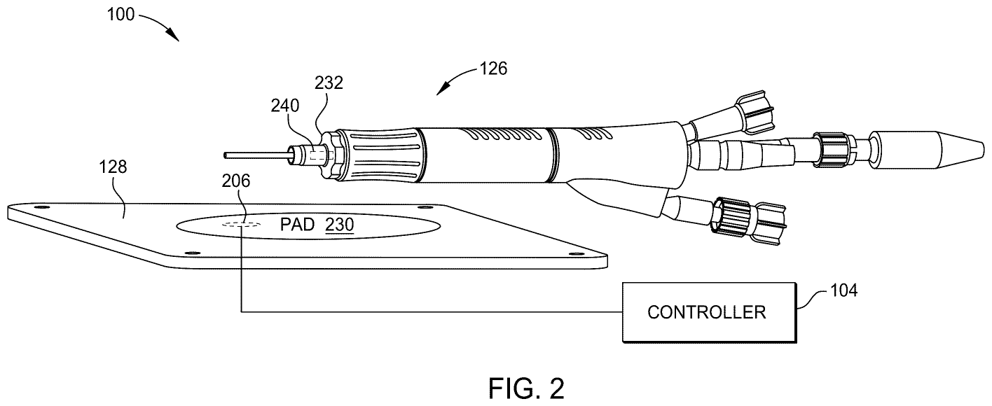

FIG. 2 illustrates certain aspects of the operating environment 100 in greater detail. In the example of FIG. 2, the surgical tool 126 is an ophthalmic handpiece having a reusable surgical device 232 as an attachment thereto. The reusable surgical device 232 is shown to be a removable component of the surgical tool 126 that, in a typical embodiment, is reusable in multiple surgical procedures in accordance with medical standards or practices.

The surgical tray 128 is shown to have a surgical pad 230 in proximity thereto (e.g., on top), such that the surgical tool 126 can be placed on the surgical pad 230 in preparation for the surgical procedure. It should be appreciated that the surgical pad 230 can be any suitable size, shape, or layout relative to the surgical tray 128. In some embodiments, the surgical pad 230 can be omitted and have its components implemented in other equipment, such as directly in the surgical tray 128, and/or the like. In certain embodiments, the surgical pad 230, the surgical tray 128, and/or the surgical console 120 can include a usage sensor configured to detect placement of the surgical tool 126 on the surgical pad 230. For example, the usage sensor can be a laser sensor, weight sensor, or other type of similar device that is positioned and configured to detect placement of the surgical tool 126 on the surgical pad 230, thereby indicating to the controller 104 an intent to use the surgical tool 126, inclusive of the reusable surgical device 232 connected thereto.

The surgical pad 230 includes, or is associated with, a reader 206 in communication with the controller 104, while the reusable surgical device 232 includes, or is associated with, a transponder 240. The reader 206 can include any suitable interface for non-contact, wireless communication (e.g., one-way or two-way signals) between the controller 104 and, e.g., the transponder 240. For example, the reader 206 may be, or include, an RFID reader, an NFC reader, or another similar wireless-type reader or receiver. In similar fashion, the transponder 240 may be, or include, a passive or active RFID or NFC transponder, sometimes referred to as a tag, or another similar device for transmitting (i.e., communicating) device-specific profile data to the reader 206. In some cases, the reader 206 and the transponder 240 may be configured for ultra high frequency (UHF) RFID communication. For illustrative purposes, examples may be periodically described herein in which the reader 206 and the transponder 240 are configured for RFID communication.

In more detail, the transponder 240 can store, in its memory, profile data about the reusable surgical device 232. The profile data can include various device-specific parameters such as, for example, a device identifier, a usage value indicating an amount of usage of the reusable surgical device 232, a manufacturing date, a manufacturing lot identifier, a manufactured power output, and/or other information. In various embodiments, the profile data stored by the transponder 240 can be updated by the controller 104 via the reader 206. One skilled in the art will appreciate that the transponder 240 can be associated with the reusable surgical device 232 in any suitable fashion, including being embedded, positioned, and/or mounted in or on any suitable location relative to the reusable surgical device 232. In the illustrated embodiment, the transponder 240 is shown to be embedded within the reusable surgical device 232.

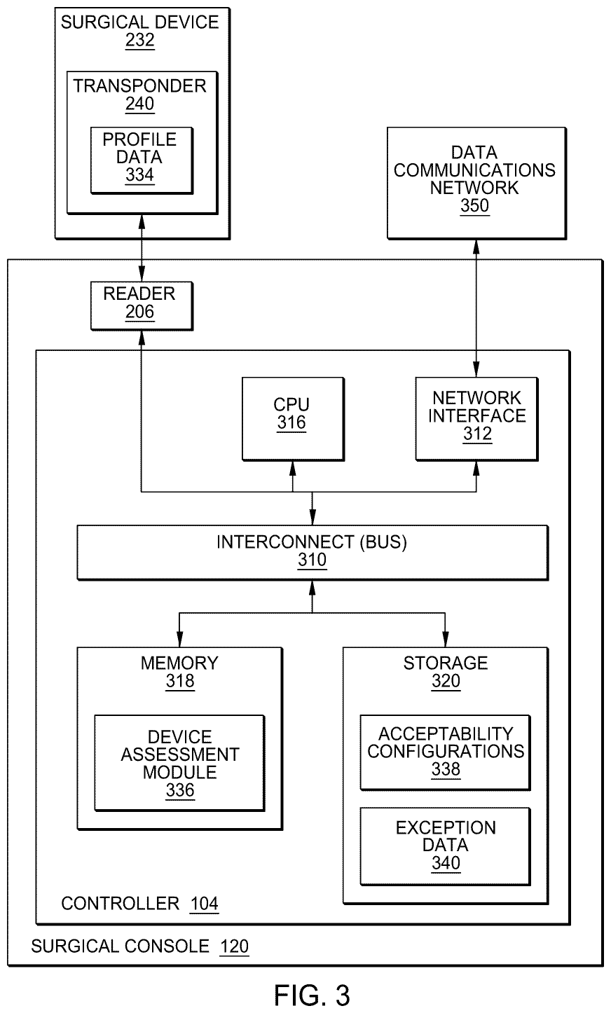

Referring to FIGS. 1-2 collectively, during performance of an ophthalmic surgical procedure on the patient 112, the surgeon 110 may utilize one or more surgical devices, including the surgical tool 126, which tool includes the reusable surgical device 232 as described previously. In certain embodiments, the controller 104 is operable to monitor utilization of the reusable surgical device 232, as well as the utilization of other similarly configured surgical devices, using the profile data stored in the transponder 240. For example, in certain embodiments, prior to the performance of the ophthalmic procedure, the controller 104 can automatically determine the acceptability (e.g., safety and/or utility) of the reusable surgical device 232 for the ophthalmic procedure and deny usage of the reusable surgical device 232 if the reusable surgical device 232 is deemed unacceptable. Further, in various embodiments, the controller 104 is operable to update the profile data stored in the transponder 240 to reflect additional usage of the reusable surgical device 232. Examples of monitoring utilization of surgical devices will be described in greater detail relative to FIG. 3-5.

FIG. 3 illustrates an example diagram showing how various components of the operating environment 100, shown in FIGS. 1-2, communicate and operate together. As shown, the transponder 240 associated with the reusable surgical device 232 includes profile data 334 in its memory. The profile data 334 can include any parameter or combination of parameters descriptive of the reusable surgical device 232 such as, for example, a usage value indicating an amount of previous usage of the reusable surgical device 232, a manufacturing date, a manufacturing lot identifier, a manufactured power output, and/or the like.

In various embodiments, at least a portion of the profile data 334 is updatable. For example, the usage value mentioned above can be adjusted or updated in response to further usage of the reusable surgical device 232. For example, if the usage value is reflective of a number of uses, the usage value can be an integer that begins at zero and is incremented with each additional surgical use of the reusable surgical device 232. Alternatively, in cases where the usage value is reflective of a number of uses, the usage value can be an integer that begins at predetermined upper boundary (e.g., ten) and is decremented with each additional surgical use of the reusable surgical device 232. In other examples, the usage value can be reflective of other indicators of use, such as time in use, such that it is either increased or decreased, as appropriate, in proportion to an amount of time each additional surgical use.

As shown, the surgical console 120 includes, without limitation, the controller 104 and the reader 206, which enable connection of the controller 104 to the transponder 240 of the reusable surgical device 232. The controller 104 includes an interconnect 310 and a network interface 312 for connection with data communications network 350. The controller 104 further includes a central processing unit (CPU) 316, memory 318, and storage 320.

The CPU 316 may retrieve and store application data in the memory 318, as well as retrieve and execute instructions stored in the memory 318. The interconnect 310 transmits programming instructions and application data among the CPU 316, the network interface 312, the memory 318, the storage 320, and the reusable surgical device 232. The CPU 316 can represent a single CPU, multiple CPUs, a single CPU having multiple processing cores, and the like. The memory 318 represents random access memory. The storage 320 may be a disk drive. Although shown as a single unit, storage 320 may be a combination of fixed or removable storage devices, such as fixed disc drives, removable memory cards or optical storage, network attached storage (NAS), or a storage area-network (SAN).

The storage 320 may include acceptability configurations 338 usable to determine acceptability of surgical devices, such as the reusable surgical device 232, for surgical procedures. In various embodiments, the acceptability configurations 338 can include, for example, one or more sets of criteria for determining safety, effectiveness, utility and/or the like based on device-specific profile data such as the profile data 334 for the reusable surgical device 232. In various embodiments, the acceptability configurations 338 can include different configurations for different types of surgical devices, different models of surgical devices, different surgical procedures, and/or the like.

For example, with respect to the reusable surgical device 232, the acceptability configurations 338 can include usage criteria such as a usage threshold associated with a usage limit of the reusable surgical device 232. The usage threshold can conform to a format or type of the usage value in the profile data 334. In an example, if the usage value is increased with each use, the usage threshold may correspond to an upper boundary that is defined in terms of a number of uses, total time in use, etc., as appropriate. In another example, if the usage value is decreased with each use, the usage threshold may correspond to a predetermined lower boundary such as zero. In some cases, the acceptability configurations 338 can include a combination of different thresholds for a variety of different usage values potentially provided in the profile data 334.

By way of further example, with respect to the reusable surgical device 232, the acceptability configurations 338 can include shelf-life criteria such as a shelf-life threshold associated with a shelf-life limit of the reusable surgical device 232. In general, the shelf-life threshold is usable in combination with at least a portion of the profile data 334, such as the manufacturing date, to evaluate shelf life. The shelf-life threshold can be specified in any suitable fashion. For example, the shelf-life threshold can be an amount of time, such that devices manufactured longer ago than that amount of time should not be used (e.g., more than 90 days ago). In another example, the shelf-life threshold can be a specified date, such that devices manufactured prior to the specified date should not be used (e.g., prior to Jan. 1, 2023).

In some cases, certain of the acceptability configurations 338 can be specified in terms of a parameter of the profile data 334, with such configuration(s) contemplating an availability of real-time data via other processes of the surgical console 120. For example, the acceptability configurations 338 can include power criteria such as a target power output, where the target power output is a reference to the manufactured power output, for example, from the profile data 334. Evaluation of the target power output may rely on, for example, a measured power output supplied by a calibration process performed prior to, or in parallel with, acceptability assessment. In various cases, the target power output can correspond to the manufactured power output, or can be a range that is defined relative to the manufactured power output. The target power output may also be defined in another suitable fashion.

In certain embodiments, the storage 320 may include exception data 340 that serves as a device blacklist. For example, the exception data 340 can identify surgical devices that should be automatically deemed unacceptable for use in all surgical procedures or certain types of surgical procedures. Such surgical devices can be identified, for example, by manufacturer, manufacturing date, manufacturing lot identifier or the like. The exception data 340 can be based on, for example, product recalls, safety notices, guidelines, or the like.

Memory 318 may include an operating system and/or one or more applications that, when executed by CPU 316, operate surgical console 120. As shown, the memory 318 includes a device assessment module 336. In various embodiments, the device assessment module 336, when executed by the CPU 316, monitors utilization of surgical devices.

More particularly, in certain embodiments, the device assessment module 336 monitors the operating environment 100 for an indication of an intent to use a surgical device, such as the reusable surgical device 232, in a surgical procedure. When such an indication is received, for example, as a result of the reusable surgical device 232 being placed on the surgical pad 230, the device assessment module 336, via the reader 206, reads the profile data 334 and assesses an acceptability of the reusable surgical device 232 according to the acceptability configurations 338 and/or the exception data 340. In general, the device assessment module 336 can establish that the reusable surgical device 232 is acceptable for use in the surgical procedure when there is no basis for deeming it unacceptable.

In various embodiments, the device assessment module 336 can establish that the reusable surgical device 232 is unacceptable for use in the surgical procedure based on the acceptability configurations 338. For example, the device assessment module 336 can compare the usage value from the profile data 334 to the usage threshold from the acceptability configurations 338 and determine that the usage threshold has been reached. In another example, the device assessment module 336 can compare manufacturing information from the profile data 334, such as the manufacturing date, to the shelf-life threshold from the acceptability configurations 338, and determine that the shelf-life threshold has been reached. In yet another example, the device assessment module 336 can compare a measured power output of the reusable surgical device 232 to the target power output defined by the acceptability configurations 338 and determine that the target power output is not satisfied, for example, due to a discrepancy between the manufactured power output and the measured power output. In various embodiments, any of the foregoing determinations may result in the reusable surgical device 232 being established as unacceptable for use in the surgical procedure.

In addition, or alternatively, the device assessment module 336 can establish that the reusable surgical device 232 is unacceptable for use in the surgical procedure based on the exception data 340. As mentioned previously, the exception data 340 may include, for example, recall data that is accessible to the device assessment module 336. For example, the device assessment module 336 can compare manufacturing information from the profile data 334, such as the manufacturing lot identifier, to the exception data 340 and determine that at least a portion of the exception data 340 is satisfied. For example, it may be determined that the manufacturing lot identifier matches an identifier for manufacturing lot that has been recalled. In various embodiments, a determination that the reusable surgical device satisfies or matches any of the exception data 340 may result the reusable surgical device 232 being established as unacceptable for use in the surgical procedure.

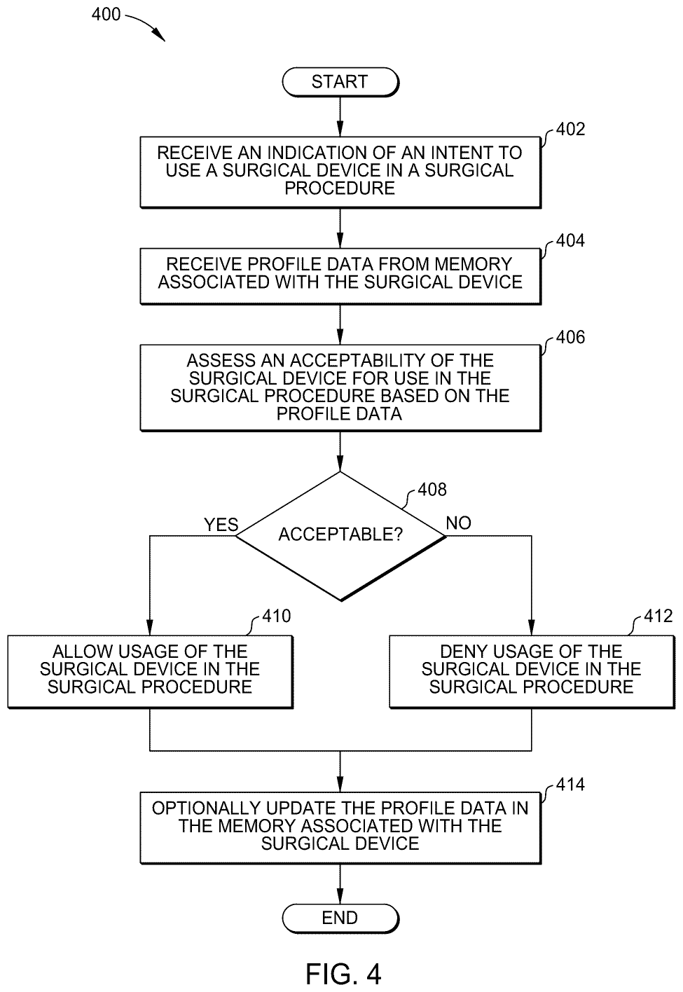

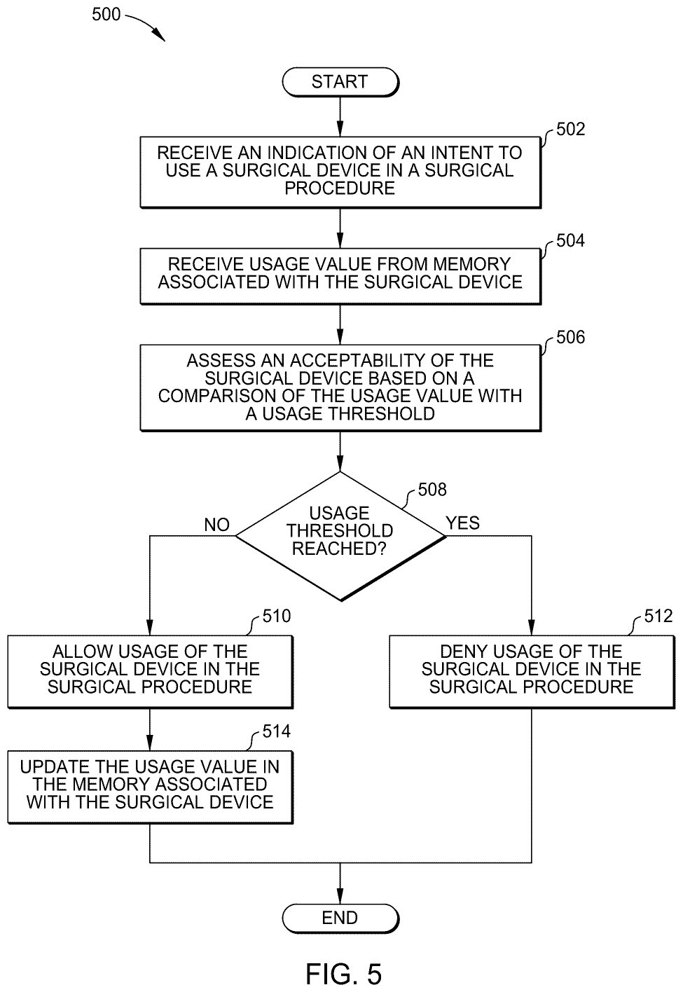

In certain embodiments, the device assessment module 336 allows usage of the reusable surgical device 232 in the surgical procedure in response to a determination that the reusable surgical device 232 is acceptable. Conversely, in a typical embodiment, the device assessment module 336 denies usage of the reusable surgical device 232 in the surgical procedure in response to a determination that the reusable surgical device 232 is unacceptable. In certain embodiments, the device assessment module 336 can update the profile data 334 in conjunction with allowing or denying usage of the reusable surgical device 232. Example operability of the device assessment module 336 to allow or deny usage of the reusable surgical device 232 and update the profile data 334 will be described in greater detail relative to FIGS. 4 and 5.

FIG. 4 illustrates an example of a process 400 for monitoring utilization of surgical devices. In certain embodiments, the process 400 can be implemented by any system that can process data. Although any number of systems, in whole or in part, can implement the process 400, to simplify discussion, the process 400 will be described in relation to example components of the operating environment 100 described relative to FIGS. 1-3.

At block 402, the device assessment module 336 receives an indicator of an intent to use the reusable surgical device 232 in a surgical procedure. The indicator can be any suitable trigger such as, for example, detection of placement of the surgical tool 126 on the surgical pad 230. In some cases, the indicator can be supplied by a user in conjunction with the user placing the surgical tool 126 on the surgical pad 230. Further, in some embodiments, the indicator can be received from another process of the surgical console 120, such as from a calibration process that executes in parallel to the process 400.

At block 404, in response to the received indicator, the device assessment module 336 receives, from memory associated with the reusable surgical device 232, the profile data 334. The block 404 can involve, for example, causing the reader 206 to read the profile data 334 from the transponder 240, for example, by interrogating the transponder 240. As mentioned previously, the profile data 334 can include, for example, a device identifier, a usage value indicating an amount of usage of the reusable surgical device 232, a manufacturing date, a manufacturing lot identifier, a manufactured power output, and/or other information.

At block 406, the device assessment module 336 assesses an acceptability of the reusable surgical device 232 for use in the surgical procedure. The assessment can be based on a comparison of the profile data 334 with the acceptability configurations 338 and/or the exception data 340. In general, the block 406 can involve the device assessment module 336 establishing that the reusable surgical device is acceptable or unacceptable in any of the ways described relative to FIG. 3.

At decision block 408, the device assessment module 336 determines whether the reusable surgical device 232 has been established as acceptable for use in the surgical procedure. If the reusable surgical device 232 has been established as acceptable, the process 400 proceeds to block 410. At block 410, the device assessment module 336 allows usage of the reusable surgical device 232 in the surgical procedure. The allowance can include, for example, advising or informing a user of the surgical console 120 in any suitable fashion (e.g., via the display device 122), recording the allowance, permitting a next step in a workflow to proceed, or the like. In embodiments in which the user is advised or informed of the allowance, the advisement can further include any information received or generated during the process 400. After block 410, the process 400 proceeds to block 414.

If it is determined, at the decision block 408, that the reusable surgical device 232 has been established as unacceptable for use in the surgical procedure, the process 400 proceeds to block 412. At block 412, the device assessment module 336 denies usage of the reusable surgical device 232 in the surgical procedure. The denial can include taking one or more actions to prevent usage of the reusable surgical device 232. For example, the denial can include advising or informing the user of the surgical console 120, aborting other processes of the surgical console 120 (e.g., a calibration process relative to the reusable surgical device 232), notifying one or more designated users inside or outside the surgical environment, halting a current workflow, initiating a different exceptional workflow, and/or the like. From block 412, the process 400 proceeds to block 414.

At block 414, the device assessment module 336 optionally updates the profile data 334 in the transponder 240 based on operation of the process 400. In general, the block 414 can involve the device assessment module 336 causing the reader 206 to write the updates to the transponder 240. In certain embodiments, if there are no updates to make, the block 414 can be omitted. In various cases, the device assessment module 336 can update the profile data 334 in response to acceptability and/or unacceptability of the reusable surgical device 232.

For example, if usage of the reusable surgical device 232 in the surgical procedure is allowed, the usage value can be updated as will be described in greater detail relative to FIG. 5. In another example, if usage of the reusable surgical device 232 is denied for certain safety-related reasons, such as due to the exceptional data 340 or due to a discrepancy between the manufactured power output and the target power output, the block 414 can involve updating at least one value in the profile data 334 to reflect that safety-related reason. In an example, the usage value can be set to the usage threshold so that no further uses will be allowed. In another example, the usage value can be set to a negative number to indicate a general safety issue. In still another example, the usage value can be set to a particular negative number indicative of the specific safety-related reason. Other examples will be apparent to one skilled in the art after a detailed review of the present disclosure. After block 414, the process 400 ends.

FIG. 5 illustrates an example of a process 500 for monitoring utilization of surgical devices via usage values. In certain embodiments, the process 500 can be implemented by any system that can process data. Although any number of systems, in whole or in part, can implement the process 400, to simplify discussion, the process 500 will be described in relation to example components of the operating environment 100 described relative to FIGS. 1-3.

At block 502, the device assessment module 336 receives an indicator of an intent to use the reusable surgical device 232 in a surgical procedure. The indicator can be any suitable trigger such as, for example, detection of placement of the surgical tool 126 on the surgical pad 230. In some cases, the indicator can be supplied by a user in conjunction with the user placing the surgical tool 126 on the surgical pad 230. Further, in some embodiments, the indicator can be received from another process of the surgical console 120, such as from a calibration process that executes in parallel to the process 400.

At block 504, in response to the received indicator, the device assessment module 336, receives, from memory associated with the reusable surgical device 232, a usage value associated with an amount of previous usage of the reusable surgical device 232. The block 404 can involve, for example, causing the reader 206 to read the usage value from the profile data 334 on the transponder 240, for example, by interrogating the transponder 240. The usage value can be specified in any of the formats described relative to FIG. 3.

At block 506, the device assessment module 336 assesses an acceptability of the reusable surgical device 232 for use in the surgical procedure. The assessment can be based on a comparison of the usage value with the usage criteria specified in the acceptability configurations 338. The usage criteria can be, or include, a usage threshold associated with a usage limit of the reusable surgical device 232. In general, the block 506 can involve the device assessment module 336 establishing that the reusable surgical device is acceptable or unacceptable based on the usage threshold, for example, in any of the ways described relative to FIG. 3.

At decision block 508, the device assessment module 336 determines whether the usage threshold has been reached. If the usage threshold has been reached, such that the reusable surgical device 232 has been established as unacceptable for use, the process 500 proceeds to block 512. At block 512, the device assessment module 336 denies usage of the reusable surgical device 232 in the surgical procedure. The denial can include taking one or more actions to prevent usage of the reusable surgical device 232. For example, the denial can include advising or informing a user of the surgical console 120 that the usage threshold has been reached, aborting other processes of the surgical console 120 (e.g., a calibration process relative to the reusable surgical device 232), notifying one or more designated users inside or outside the surgical environment, halting a current workflow, initiating a different exceptional workflow, and/or the like. From block 512, the process 500 ends.

If it is determined, at the decision block 508, that the usage threshold has not been reached, such that reusable surgical device 232 has been established as acceptable for use, the process 500 proceeds to block 510. At block 510, the device assessment module 336 allows usage of the reusable surgical device 232 in the surgical procedure. The allowance can include, for example, advising or informing a user of the surgical console 120 in any suitable fashion (e.g., via the display device 122), recording the allowance, permitting a next step in a workflow to proceed, or the like. In embodiments in which the user is advised or informed of the allowance, the advisement can further include any information received or generated during the process 400. In addition, or alternatively, the advisement can include, for example, a number of remaining uses of the reusable surgical device 232, or indicating if the allowed usage will be the final usage due to the usage threshold having now been reached.

At block 514, the device assessment module 336 updates the usage value in the memory associated with the reusable surgical device 232 to reflect the additional allowed usage. In general, the block 414 can involve the device assessment module 336 causing the reader 206 to write the update to the profile data 334 on the transponder 240. For example, the device assessment module 336 can increase or decrease the usage value in any of the ways described relative to FIG. 3 to reflect the allowed usage. After block 514, the process 500 ends.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A method of monitoring utilization of reusable surgical devices in a surgical operating environment, comprising, by a surgical console:
receiving an indicator of an intent to use a reusable surgical device in a surgical procedure;
responsive to the received indicator, receiving, from memory associated with the reusable surgical device, a usage value associated with an amount of previous usage of the reusable surgical device;
assessing an acceptability of the reusable surgical device for use in the surgical procedure based on a comparison of the usage value with a usage threshold associated with a usage limit of the reusable surgical device; and
allowing or denying usage of the reusable surgical device in the surgical procedure based on the assessed acceptability of the reusable surgical device.

2. The method of claim 1, wherein:
the assessing comprises establishing that the reusable surgical device is unacceptable for use in the surgical procedure based on a determination that the usage value has reached the usage threshold; and
the allowing or denying comprises denying usage of the reusable surgical device in the surgical procedure responsive to the determination that the reusable surgical device is unacceptable.

3. The method of claim 1, wherein:
the assessing comprises establishing that the reusable surgical device is acceptable for use in the surgical procedure based on a determination that the usage value has not reached the usage threshold associated with the usage limit of the reusable surgical device; and
the allowing or denying comprises allowing usage of the reusable surgical device in the surgical procedure responsive to the establishment that the reusable surgical device is acceptable.

4. The method of claim 3, the method further comprising updating, by the surgical console, the usage value in the memory associated with the reusable surgical device to reflect the allowed usage.

5. The method of claim 4, wherein the updating comprises decreasing the usage value.

6. The method of claim 1, wherein the allowing or denying comprises advising a user of the allowed or denied usage.

7. The method of claim 1, wherein the reusable surgical device is an attachment to a surgical tool.

8. The method of claim 1, wherein the receiving an indicator comprises detecting placement of the reusable surgical device in proximity to a surgical tray of the surgical console.

9. The method of claim 1, further comprising, responsive to the received indicator, receiving, by the surgical console, from the memory associated with the reusable surgical device, manufacturing information for the reusable surgical device, wherein:
the assessing an acceptability of the reusable surgical device is further based on a comparison of the manufacturing information with a shelf-life threshold associated with a shelf-life limit of the reusable surgical device;
the assessing comprises establishing that the reusable surgical device is unacceptable for use in the surgical procedure based on a determination that the shelf-life threshold has been reached; and
the allowing or denying comprises denying usage of the reusable surgical device in the surgical procedure responsive to the determination that the reusable surgical device is unacceptable.

10. The method of claim 1, further comprising, responsive to the received indicator, receiving, by the surgical console, from the memory associated with the reusable surgical device, manufacturing information for the reusable surgical device, wherein:
the assessing an acceptability of the reusable surgical device is further based on a comparison of the manufacturing information with recall data accessible to the surgical console.
the assessing comprises establishing that the reusable surgical device is unacceptable for use in the surgical procedure based on a determination that the manufacturing information matches at least a portion of the recall data; and
the allowing or denying comprises denying usage of the reusable surgical device in the surgical procedure responsive to the determination that the reusable surgical device is unacceptable.

11. The method of claim 1, further comprising, responsive to the received indicator, receiving, by the surgical console, from the memory associated with the reusable surgical device, a manufactured power output for the reusable surgical device, wherein:
the assessing an acceptability of the reusable surgical device is further based on a comparison of a measured power output of the reusable surgical device with the manufactured power output;
the assessing comprises establishing that the reusable surgical device is unacceptable for use in the surgical procedure based on a determination of a discrepancy between the manufactured power output and the measured power output; and
the allowing or denying comprises denying usage of the reusable surgical device in the surgical procedure responsive to the determination that the reusable surgical device is unacceptable.

12. The method of claim 11, further comprising updating, by the surgical console, at least one value in the memory associated with the reusable surgical device to reflect the discrepancy.

13. A system for monitoring utilization of reusable surgical devices in a surgical operating environment, the system comprising:
a reader; and
a surgical console communicably coupled to the reader, wherein the surgical console is operable to:
receive an indicator of an intent to use a reusable surgical device in a surgical procedure;
responsive to the received indicator, receive, via the reader, from memory associated with the reusable surgical device, a usage value associated with an amount of previous usage of the reusable surgical device;
assess an acceptability of the reusable surgical device for use in the surgical procedure based on a comparison of the usage value with a usage threshold associated with a usage limit of the reusable surgical device; and
allow or deny usage of the reusable surgical device in the surgical procedure based on the assessed acceptability of the reusable surgical device.

14. The system of claim 13, further comprising a surgical tray, wherein the reader is a radio frequency identification (RFID) reader and is positioned in proximity to the surgical tray.

15. The system of claim 14, further comprising a surgical pad in proximity to the surgical tray, wherein the reader is embedded in the surgical pad.
